(19) United States
(12) Patent Application Publication
(10) Pub. No.: US 2007/0208426 A1
(43) Pub. Date: Sep. 6, 2007

(54) Spinal implant with improved surface properties for delivery

(75) Inventor: Hai H. Trieu, Cordova, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: SDGI HOLDINGS, INC., Wilmington, DE (US)

(21) Appl. No.: 11/368,126

(22) Filed: Mar. 3, 2006

(51) Int. Cl. A61F 2/44; A61F 2/46

(52) U.S. Cl. 623/17.16; 623/17.14

(57) ABSTRACT

An intervertebral prosthetic disc is disclosed and can be installed within an intervertebral space between a superior vertebra and an inferior vertebra. The intervertebral prosthetic disc can include a superior component and an inferior component that can have an inferior bearing surface. A hydrophilicity of the inferior bearing surface can be greater-than an average hydrophilicity of the inferior component.

### FIELD OF THE DISCLOSURE

The present disclosure relates generally to orthopedics and spinal surgery. More specifically, the present disclosure relates to spinal implants.

### BACKGROUND

In human anatomy, the spine is a generally flexible column that can take tensile and compressive loads. The spine also allows bending motion and provides a place of attachment for keels, muscles and ligaments. Generally, the spine is divided into three sections: the cervical spine, the thoracic spine and the lumbar spine. The sections of the spine are made up of individual bones called vertebrae. Also, the vertebrae are separated by intervertebral discs, which are situated between adjacent vertebrae.

The intervertebral discs function as shock absorbers and as joints. Further, the intervertebral discs can absorb the compressive and tensile loads to which the spinal column may be subjected. At the same time, the intervertebral discs can allow adjacent vertebral bodies to move relative to each other a limited amount, particularly during bending, or flexure, of the spine. Thus, the intervertebral discs are under constant muscular and/or gravitational pressure and generally, the intervertebral discs are the first parts of the lumbar spine to show signs of deterioration.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints and/or intervertebral disc may cause spinal stenosis, degenerative spondylolisthesis, and degenerative scoliosis.

One surgical procedure for treating these conditions is spinal arthrodesis, i.e., spine fusion, which can be performed anteriorally, posteriorally, and/or laterally. The posterior procedures include in-situ fusion, posterior lateral instrumented fusion, transforaminal lumbar interbody fusion (“TLIF”) and posterior lumbar interbody fusion (“PLIF”). Solidly fusing a spinal segment to eliminate any motion at that level may alleviate the immediate symptoms, but for some patients maintaining motion may be beneficial. It is also known to surgically replace a degenerative disc or facet joint with an artificial disc or an artificial facet joint, respectively.

### BRIEF DESCRIPTION OF THE DRAWINGS

FIG. 1 is a lateral view of a portion of a vertebral column;

FIG. 2 is a lateral view of a pair of adjacent vertrebrae;

FIG. 3 is a top plan view of a vertebra;

FIG. 4 is a cross section view of an intervertebral disc;

FIG. 5 is a posterior view of a second embodiment of an intervertebral prosthetic disc;

FIG. 6 is an exploded posterior view of the second embodiment of the intervertebral prosthetic disc;

FIG. 7 is an exploded posterior view of the second embodiment of the intervertebral prosthetic disc;

FIG. 8 is a lateral view of the second embodiment of the intervertebral prosthetic disc;

FIG. 9 is an exploded lateral view of the second embodiment of the intervertebral prosthetic disc;

FIG. 10 is a plan view of a superior half of the second embodiment of the intervertebral prosthetic disc;

FIG. 11 is another plan view of the superior half of the second embodiment of the intervertebral prosthetic disc;

FIG. 12 is a plan view of an inferior half of the second embodiment of the intervertebral prosthetic disc;

FIG. 13 is another plan view of the inferior half of the second embodiment of the intervertebral prosthetic disc;

FIG. 14 is an exploded lateral view of the first embodiment of the intervertebral prosthetic disc installed within an intervertebral space between a pair of adjacent vertrebrae;

FIG. 15 is an anterior view of the first embodiment of the intervertebral prosthetic disc installed within an intervertebral space between a pair of adjacent vertrebrae;

FIG. 16 is a plan view of a nucleus implant installed within an intervertebral disc;

FIG. 17 is a plan view of the nucleus implant within an implant delivery device;

FIG. 18 is a plan view of the nucleus implant exiting the implant delivery device;

FIG. 19 is a cross-section view of the nucleus implant;

FIG. 20 is a cross-section view of the implant delivery device;

FIG. 21 is a flow chart of a method of installing a spinal implant; and

FIG. 22 is a flow chart of another method of installing a spinal implant.

### DETAILED DESCRIPTION OF THE DRAWINGS

An intervertebral prosthetic disc is disclosed and can be installed within an intervertebral space between a superior vertebra and an inferior vertebra. The intervertebral prosthetic disc can include a superior component and an inferior component that can have an inferior bearing surface. A hydrophilicity of the inferior bearing surface can be greater than an average hydrophilicity of the inferior component.

In a particular embodiment, increasing the hydrophilicity of the inferior bearing surface, or any other surface, can increase the wettability of that surface. Further, increasing the wettability can increase the lubricity of the surface when wetted and can reduce friction, which can be beneficial during delivery or implantation of the intervertebral prosthetic disc.

In another embodiment, a nucleus implant is disclosed and can be installed within an intervertebral space within an intervertebral disc. The nucleus implant can include a load bearing elastic body movable between a folded configuration and a substantially straight configuration. The load bearing elastic body can include a core and an outer hydrophilic layer around the core.

In yet another embodiment, a method of installing a spinal implant having a hydrophilic surface is disclosed. The method can include exposing the hydrophilic surface to a fluid and installing the spinal implant.

In still another embodiment, a method of installing a spinal implant having a hydrophilic layer is disclosed. The method can include exposing the hydrophilic layer to a fluid and installing the spinal implant.

In yet still another embodiment, a method of installing a spinal implant having a hydrophilic surface is disclosed. The method can include soaking the spinal implant in a fluid for a predetermined time and installing the spinal implant.

In another embodiment, a method of installing a spinal implant having a hydrophilic surface is disclosed. The method can include soaking the spinal implant in a fluid for a predetermined time, retrieving the spinal implant from the fluid, and installing the spinal implant.

In still another embodiment, a method of installing a spinal implant is disclosed and includes soaking the spinal implant in a fluid for a predetermined time to increase the lubrication of the spinal implant and soaking an implant delivery device in the fluid for a predetermined time to increase the lubrication of the implant delivery device.

In yet another embodiment, an implant delivery device is disclosed and includes a housing that can have an outer structure and an inner hydrophilic layer thereon.

In yet still another embodiment, a method of installing a spinal implant is disclosed and includes decreasing a coefficient of friction of the spinal implant and installing the implant.

In another embodiment, a spinal implant is disclosed and can be installed between a superior vertebra and an inferior vertebra. The spinal implant can include a component that can have a surface that can contact an interior surface of a delivery device, human tissue, or a combination thereof during installation. The surface of the component can have a hydrophilicity that is greater than an average hydrophilicity of an underlying material of the component.

Description of Relevant Anatomy

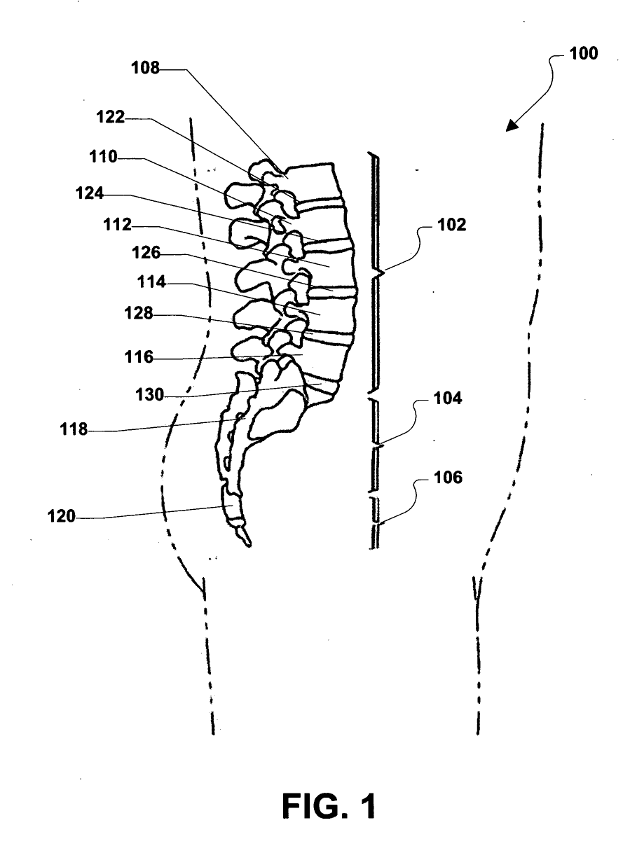

Referring initially to FIG. 1, a portion of a vertebral column, designated 100, is shown. As depicted, the vertebral column 100 includes a lumbar region 102, a sacral region 104, and a coccygeal region 106. As is known in the art, the vertebral column 100 also includes a cervical region and a thoracic region. For clarity and ease of discussion, the cervical region and the thoracic region are not illustrated.

As shown in FIG. 1, the lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumbar vertebra 108 and the second lumbar vertebra 110. A second intervertebral lumbar disc 124, is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

In a particular embodiment, if one of the intervertebral lumbar discs 122, 124, 126, 128, 130 is diseased, degenerated, damaged, or otherwise in need of replacement, that intervertebral lumbar disc 122, 124, 126, 128, 130 can be at least partially removed and replaced with an intervertebral prosthetic disc according to one or more of the embodiments described herein. In a particular embodiment, a portion of the intervertebral lumbar disc 122, 124, 126, 128, 130 can be removed via a discectomy, or a similar surgical procedure, well known in the art. Further, removal of intervertebral lumbar disc material can result in the formation of an intervertebral space (not shown) between two adjacent lumbar vertebrae.

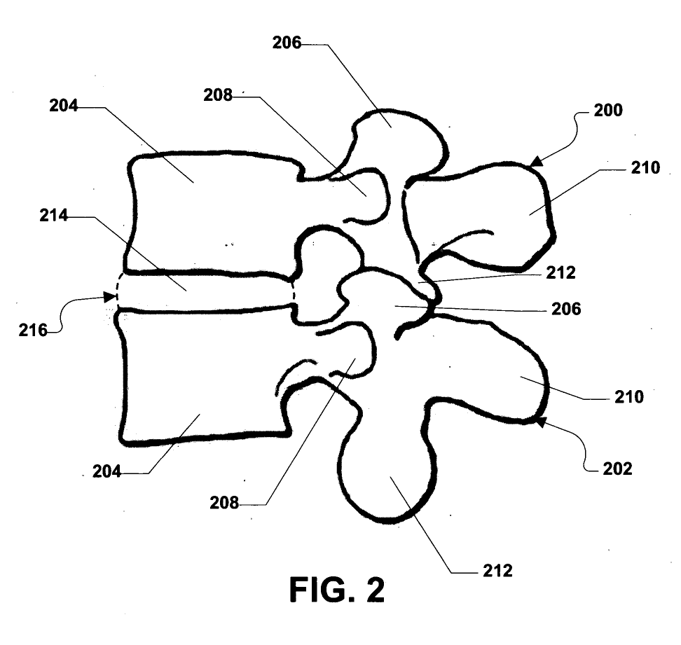

FIG. 2 depicts a detailed lateral view of two adjacent vertebrae, e.g., two of the lumbar vertebra 108, 110, 112, 114, 116 shown in FIG. 1. FIG. 2 illustrates a superior vertebra 200 and an inferior vertebra 202. As shown, each vertebra 200, 202 includes a vertebral body 204, a superior articular process 206, a transverse process 208, a spinous process 210 and an inferior articular process 212. FIG. 2 further depicts an intervertebral space 214 that can be established between the superior vertebra 200 and the inferior vertebra 202 by removing an intervertebral disc 216 (shown in dashed lines). As described in greater detail below, an intervertebral prosthetic disc according to one or more of the embodiments described herein can be installed within the intervertebral space 212 between the superior vertebra 200 and the inferior vertebra 202.

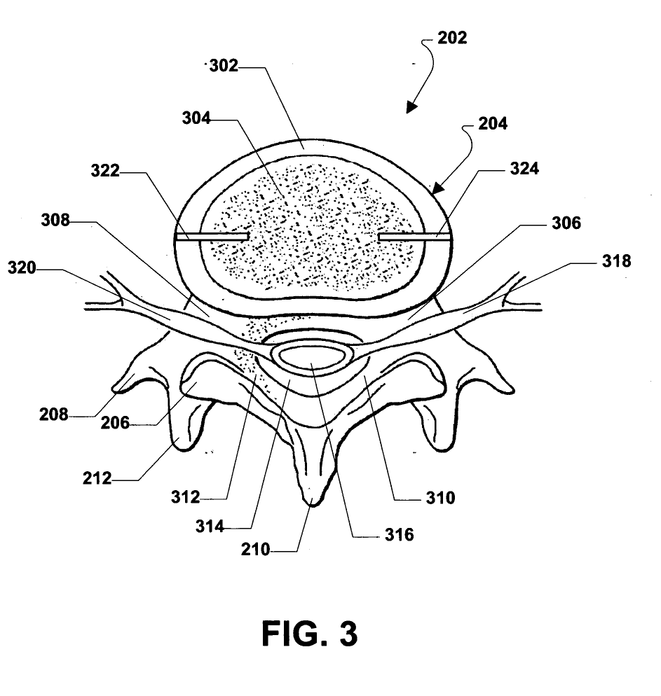

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

FIG. 3 further depicts a first slot 322 and a second slot 324 that can be established within the cortical rim 302 of the inferior vertebra 302. In a particular embodiment, the first slot 322 and the second slot 324 are established during surgery to install an intervertebral prosthetic disc according to one or more of the embodiments described herein. The first slot 322 and the second slot 324 can be established using a cutting device, e.g., a chisel that is designed to cut a groove, or slot, in a vertebra, prior to the installation of the intervertebral prosthetic disc. Further, the first slot 322 and the second slot 324 are sized and shaped to receive and engage a first rib and a second rib, described in detail below, that extend from an intervertebral prosthetic disc according to one or more of the embodiments described herein. The first slot 322 and the second slot 324 can cooperate with a first rib and second rib to facilitate proper alignment of an intervertebral prosthetic disc within an intervertebral space between an inferior vertebra and a superior vertebra.

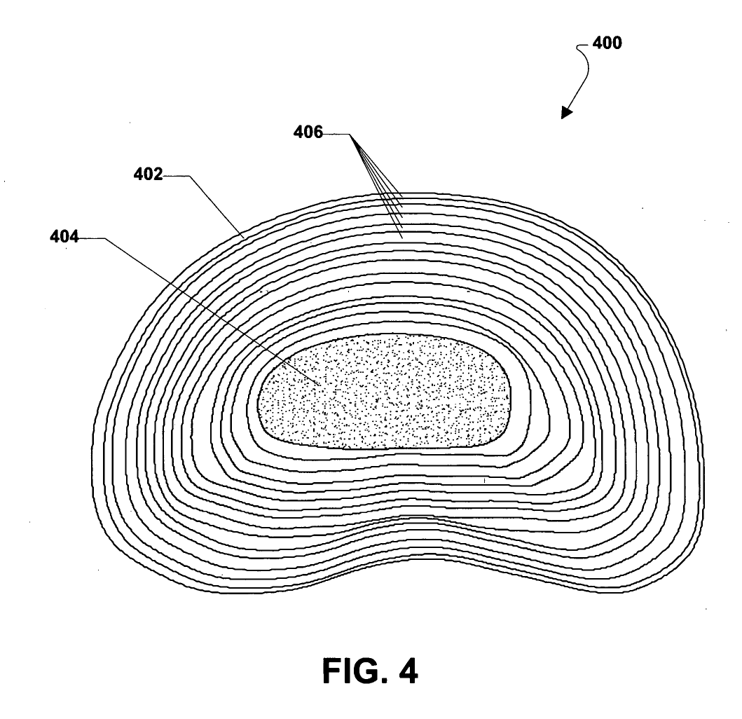

Referring now to FIG. 4, an intervertebral disc is shown and is generally designated 400. The intervertebral disc 400 is made up of two components: the annulus fibrosis 402 and the nucleus pulposus 404. The annulus fibrosis 402 is the outer portion of the intervertebral disc 400, and the annulus fibrosis 402 includes a plurality of lamellae 406. The lamellae 406 are layers of collagen and proteins. Each lamella 406 includes fibers that slant at 30-degree angles, and the fibers of each lamella 406 run in a direction opposite the adjacent layers. Accordingly, the annulus fibrosis 402 is a structure that is exceptionally strong, yet extremely flexible.

The nucleus pulposus 404 is the inner gel material that is surrounded by the annulus fibrosis 402. It makes up about forty percent (40%) of the intervertebral disc 400 by weight. Moreover, the nucleus pulposus 404 can be considered a ball-like gel that is contained within the lamellae 406. The nucleus pulposus 404 includes loose collagen fibers, water, and proteins. The water content of the nucleus pulposus 404 is about ninety percent (90%) by weight at birth and decreases to about seventy percent by weight (70%) by the fifth decade.

Injury or aging of the annulus fibrosis 402 may allow the nucleus pulposus 404 to be squeezed through the annulus fibers either partially, causing the disc to bulge, or completely, allowing the disc material to escape the intervertebral disc 400. The bulging disc or nucleus material may compress the nerves or spinal cord, causing pain. Accordingly, the nucleus pulposus 404 can be removed and replaced with an artificial nucleus.

Description of a First Embodiment of an Intervertebral Prosthetic Disc

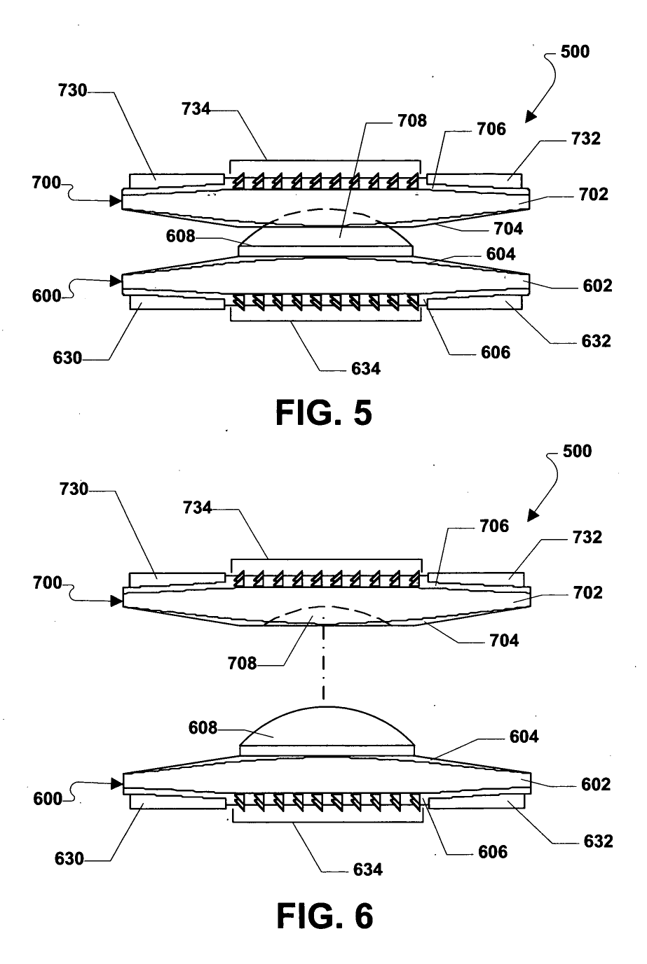

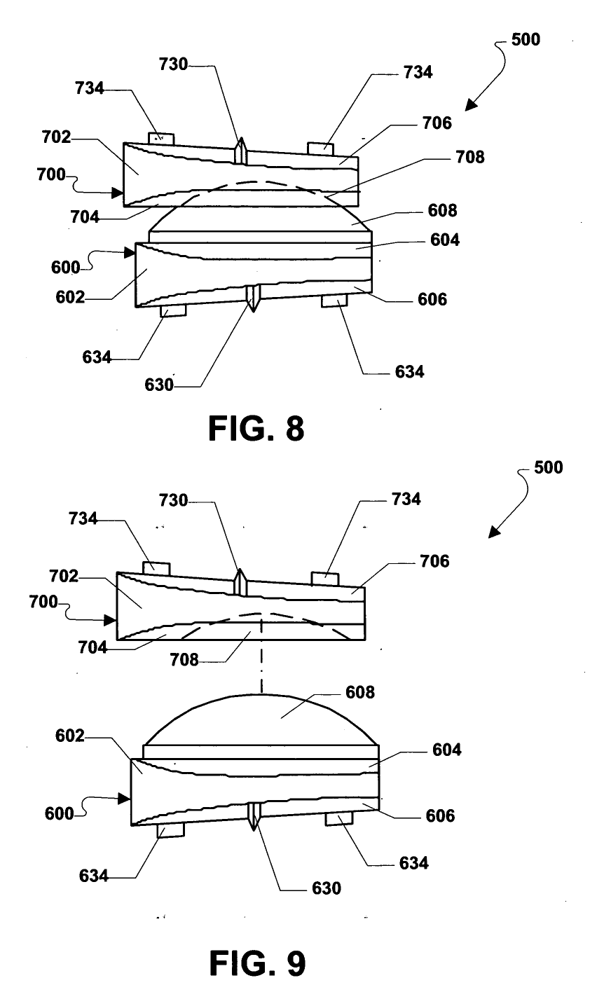

Referring to FIGS. 5 through 13 a first embodiment of an intervertebral prosthetic disc is shown and is generally designated 500. As illustrated, the intervertebral prosthetic disc 500 can include an inferior component 600 and a superior component 700. In a particular embodiment, the components 600, 700 can be made from one or more biocompatible materials. In a particular embodiment, the components 500, 600 can be made from one or more biocompatible materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyaryletherketone (PAEK) materials, silicone materials, hydrogel materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyetherketoneetherketoneketone (PEKEKK), or a combination thereof. The hydrogels can include polyacrylamide, poly-N-isopropylacrylamine, polyvinyl methylether, polyvinyl alcohol, polyethyl hydroxyethyl cellulose, poly(2-ethyl)oxazoline, polyethyleneoxide, polyethylglycol, polyethylene glycol, polyacrylic acid, polyacrylonitrile, polyvinylacrylate, polyvinylpyrrolidone, or a combination thereof. Alternatively, the components 600, 700 can be made from any other substantially rigid biocompatible materials.

In a particular embodiment, the inferior component 600 can include an inferior support plate 602 that has an inferior articular surface 604 and an inferior bearing surface 606. In a particular embodiment, the inferior articular surface 604 can be generally rounded and the inferior bearing surface 606 can be generally flat.

As illustrated in FIG. 5 through FIG. 12, a projection 608 extends from the inferior articular surface 604 of the inferior support plate 602. In a particular embodiment, the projection 608 has a hemi-spherical shape. Alternatively, the projection 608 can have an elliptical shape, a cylindrical shape, or other arcuate shape.

FIG. 5 through FIG. 9 and FIG. 11 also show that the inferior component 600 can include a first inferior keel 630, a second inferior keel 632, and a plurality of inferior teeth 634 that extend from the inferior bearing surface 606. As shown, in a particular embodiment, the inferior keels 630, 632 and the inferior teeth 634 are generally saw-tooth, or triangle, shaped. Further, the inferior keels 630, 632 and the inferior teeth 634 are designed to engage cancellous bone, cortical bone, or a combination thereof of an inferior vertebra. Additionally, the inferior teeth 634 can prevent the inferior component 600 from moving with respect to an inferior vertebra after the intervertebral prosthetic disc 500 is installed within the intervertebral space between the inferior vertebra and the superior vertebra.

In a particular embodiment, the inferior teeth 634 can include other projections such as spikes, pins, blades, or a combination thereof that have any cross-sectional geometry.

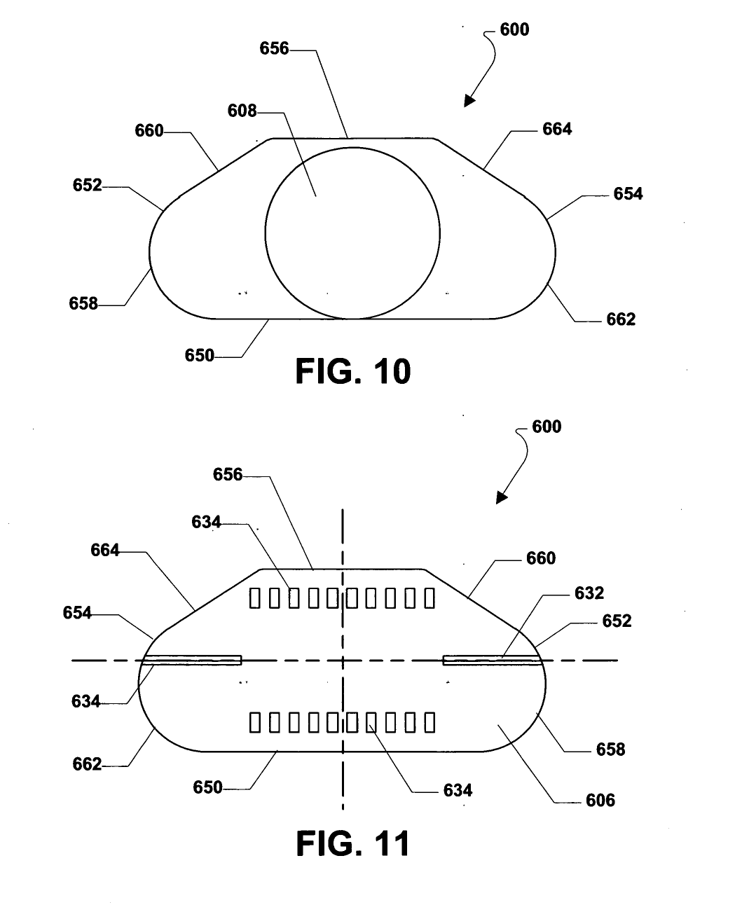

As illustrated in FIG. 10 and FIG. 11, the inferior component 600 can be generally shaped to match the general shape of the vertebral body of a vertebra. For example, the inferior component 600 can have a general trapezoid shape and the inferior component 600 can include a posterior side 650. A first lateral side 652 and a second lateral side 654 can extend from the posterior side 650 to an anterior side 656. In a particular embodiment, the first lateral side 652 can include a curved portion 658 and a straight portion 660 that extends at an angle toward the anterior side 656. Further, the second lateral side 654 can also include a curved portion 662 and a straight portion 664 that extends at an angle toward the anterior side 656.

As shown in FIG. 10 and FIG. 11, the anterior side 656 of the inferior component 600 can be relatively shorter than the posterior side 650 of the inferior component 600. Further, in a particular embodiment, the anterior side 656 is substantially parallel to the posterior side 650. As indicated in FIG. 10, the projection 608 can be situated relative to the inferior articular surface 604 such that the perimeter of the projection 608 is tangential to the posterior side 650 of the inferior component 600. In alternative embodiments (not shown), the projection 608 can be situated relative to the inferior articular surface 604 such that the perimeter of the projection 608 is tangential to the anterior side 656 of the inferior component 600 or tangential to both the anterior side 656 and the posterior side 650.

In a particular embodiment, the superior component 700 can include a superior support plate 702 that has a superior articular surface 704 and a superior bearing surface 706. In a particular embodiment, the superior articular surface 704 can be generally rounded and the superior bearing surface 706 can be generally flat.

As illustrated in FIG. 5 through FIG. 13, a depression 708 extends into the superior articular surface 704 of the superior support plate 702. In a particular embodiment, the depression 708 has a hemi-spherical shape. Alternatively, the depression 708 can have an elliptical shape, a cylindrical shape, or other arcuate shape.

FIG. 5 through FIG. 9 and FIG. 13 also show that the superior component 700 can include a first superior keel 730, a second superior keel 732, and a plurality of superior teeth 734 that extend from the superior bearing surface 706. As shown, in a particular embodiment, the superior keels 730, 732 and the superior teeth 734 are generally saw-tooth, or triangle, shaped. Further, the superior keels 730, 732 and the superior teeth 734 are designed to engage cancellous bone, cortical bone, or a combination thereof, of a superior vertebra. Additionally, the superior teeth 734 can prevent the superior component 700 from moving with respect to a superior vertebra after the intervertebral prosthetic disc 500 is installed within the intervertebral space between the inferior vertebra and the superior vertebra.

In a particular embodiment, the superior teeth 734 can include other depressions such as spikes, pins, blades, or a combination thereof that have any cross-sectional geometry.

In a particular embodiment, the superior component 700 can be shaped to match the shape of the inferior component 600, shown in FIG. 10 and FIG. 11. Further, the superior component 700 can be shaped to match the general shape of a vertebral body of a vertebra. For example, the superior component 700 can have a general trapezoid shape and the superior component 700 can include a posterior side 750. A first lateral side 752 and a second lateral side 754 can extend from the posterior side 750 to an anterior side 756. In a particular embodiment, the first lateral side 752 can include a curved portion 758 and a straight portion 760 that extends at an angle toward the anterior side 756. Further, the second lateral side 754 can also include a curved portion 762 and a straight portion 764 that extends at an angle toward the anterior side 756.

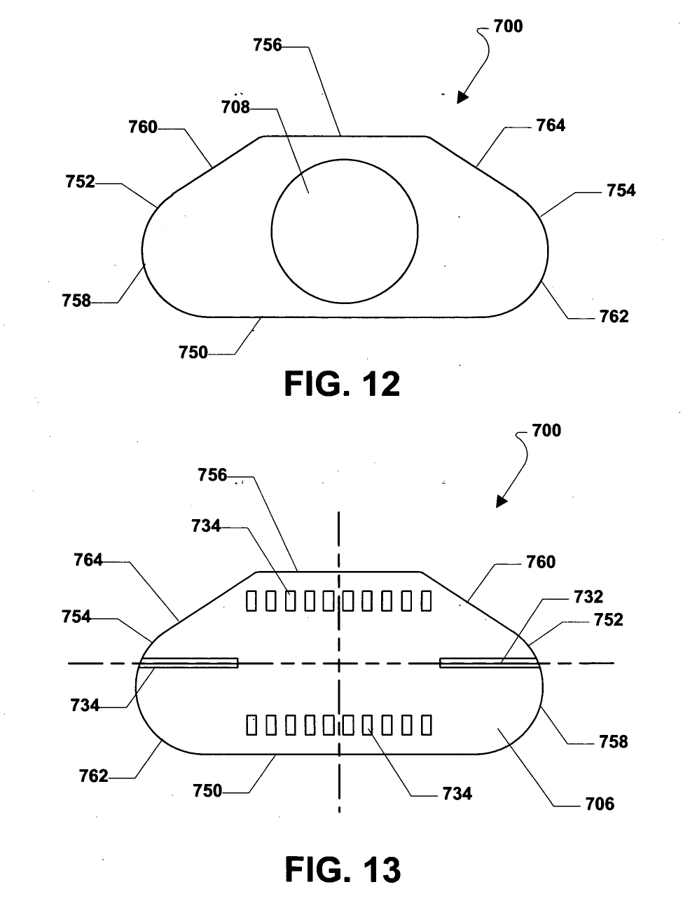

As shown in FIG. 12 and FIG. 13, the anterior side 756 of the superior component 700 can be relatively shorter than the posterior side 750 of the superior component 700. Further, in a particular embodiment, the anterior side 756 is substantially parallel to the posterior side 750.

In a particular embodiment, the overall height of the intervertebral prosthetic disc 500 can be in a range from six millimeters to twenty-two millimeters (6-22 mm). Further, the installed height of the intervertebral prosthetic disc 500 can be in a range from four millimeters to sixteen millimeters (4-16 mm). In a particular embodiment, the installed height can be substantially equivalent to the distance between an inferior vertebra and a superior vertebra when the intervertebral prosthetic disc 500 is installed there between.

In a particular embodiment, the intervertebral prosthetic disc 500 can be considered to be “low profile.” The low profile the intervertebral prosthetic disc 500 can allow the intervertebral prosthetic disc 500 to be implanted into an intervertebral space between an inferior vertebra and a superior vertebra laterally through a patient's psoas muscle, e.g., through an insertion device. Accordingly, the risk of damage to a patient's spinal cord or sympathetic chain can be substantially minimized. In alternative embodiments, all of the superior and inferior teeth 618, 718 can be oriented to engage in a direction substantially opposite the direction of insertion of the prosthetic disc into the intervertebral space.

Further, the intervertebral prosthetic disc 500 can have a general “bullet” shape as shown in the posterior plan view, described herein. The bullet shape of the intervertebral prosthetic disc 500 can further allow the intervertebral prosthetic disc 500 to be inserted through the patient's psoas muscle while minimizing risk to the patient's spinal cord and sympathetic chain.

In a particular embodiment, the length of the intervertebral prosthetic disc 500, e.g., along a longitudinal axis, can be in a range from thirty-three millimeters to fifty millimeters (33-50 mm). Additionally, the width of the intervertebral prosthetic disc 500, e.g., along a lateral axis, can be in a range from eighteen millimeters to twenty-nine millimeters (18-29 mm).

In a particular embodiment, the intervertebral prosthetic disc 500 can be treated to increase the hydrophilicity of the intervertebral prosthetic disc 500. Specifically, the external surfaces of the intervertebral prosthetic disc 500, e.g., the inferior bearing surface 606 and the superior bearing surface 706, can be treated to make those surfaces more hydrophilic than the underlying bulk material that is used to make the intervertebral prosthetic disc 500. Consequently, the hydrophilicity of the inferior bearing surface 606, the superior bearing surface 706, or a combination thereof can be greater than the average hydrophilicity of the inferior component 600 and/or the superior component 700 respectively.

For example, the hydrophilicity of the inferior bearing surface 606 and the superior bearing surface 706 can be increased by oxidizing the inferior bearing surface 606 and the superior bearing surface 706. Additionally, the hydrophilicity of the inferior bearing surface 606 and the superior bearing surface 706 can be increased by modifying the inferior bearing surface 606 and the superior bearing surface 706 using a chemical technique or an electrochemical technique.

In a particular embodiment, the chemical technique or the electrochemical technique can include a gas plasma technique. In other words, the inferior bearing surface 606 and the superior bearing surface 706 can be exposed to a gas plasma in order to modify the hydrophilicity or wettability of the inferior bearing surface 606 and the superior bearing surface 706. For example, the bearing surfaces 606, 706 can be modified using a cold gas plasma process. The cold gas plasma process can include placing the intervertebral prosthetic disc 500 in a vacuum and pumping in one or more process fluids. Radio-frequency energy can be supplied to one or more electrodes within the chamber in order to excite the process fluid into plasma.

The process fluid can include one or more gases, one or more liquids, or a combination thereof. Further, the one or more gases can include oxygen, argon, helium, nitrogen, ammonia, hydrogen, nitrous oxide, carbon dioxide, air, methane, ethane, ethylene, acetylene, tetrafluoromethane, hexafluoroethane, hexafluoropropylene, or combination thereof. Moreover, the one or more liquids can include methanol, water, allyl amine, ethylenediamine, acrylic acide, acetone, hydroxyethylmethacrylate, ethanol, toluene, diaminopropane, butylamine, gluteraldehyde, hexamethyldisiloxane, tetramethylsilane, polyethylene glycol, diglyme, silane, or a combination thereof.

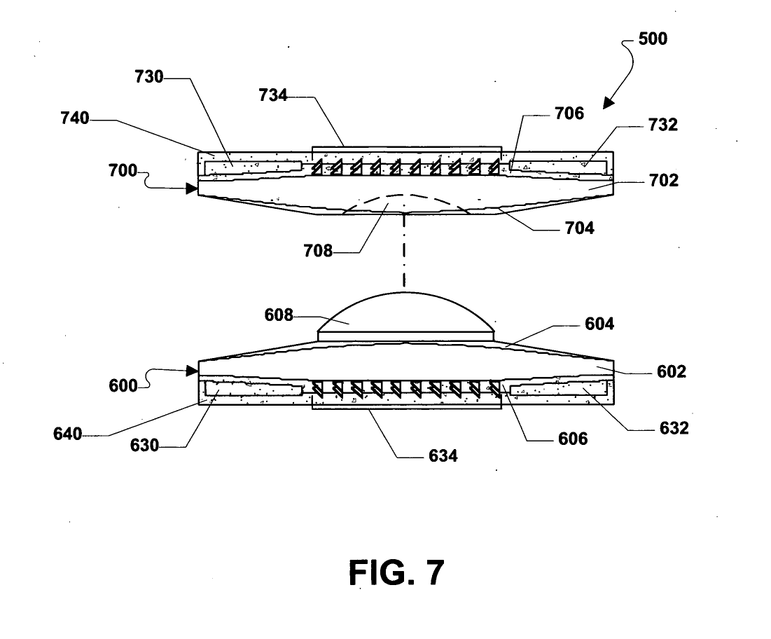

As shown in FIG. 7, the inferior bearing surface 606 can include an inferior hydrophilic layer 640 and the superior bearing surface 706 can include a superior hydrophilic layer 740. In a particular embodiment, the inferior hydrophilic layer 640 and the superior hydrophilic layer 740 can be one or more hydrophilic polymers.

The hydrophilic polymers can include polyalkylene glycol, polymethacrylates, maleic anhydride/vinyl ether copolymer, starch, starch derivatives, gelatin, alginate, hydroxyethyl methacrylate, carrageenan, polyurethane, agar, carboxyvinyl copolymer, polyethylene oxide, polyhydroxyethyl methacrylate, polydioxolane, polyacryl acetate, polyvinyl chloride, or a combination thereof.

Further, the hydrophilic polymers can include one or more cellulose derivative, such as hydroxypropylmethyl cellulose, hydroxypropyl cellulose, carboxymethyl cellulose, methyl cellulose, ethyl cellulose, hydroxyethyl cellulose, hydroxymethyl cellulose, carboxyethylcellulose, carboxy-methyl-hydroxy-ethyl cellulose, or a combination thereof.

In another particular embodiment, the inferior hydrophilic layer 640 and the superior hydrophilic layer 740 can be one or more hydrogels. The hydrogels can include polyacrylamide, poly-N-isopropylacrylamine, polyvinyl methylether, polyvinyl alcohol, polyethyl hydroxyethyl cellulose, poly(2-ethyl)oxazoline, polyethyleneoxide, polyethylglycol, polyethylene glycol, polyacrylic acid, polyacrylonitrile, polyvinylacrylate, polyvinylpyrrolidone, or a combination thereof.

In a particular embodiment, the inferior hydrophilic layer 640 and the superior hydrophilic layer 740 can be resorbable, non-resorbable, temporary, permanent, semi-permanent, detachable, removable, or a combination thereof. For example, the hydrophilic layers 640, 740 may provide lubrication during installation, e.g., during delivery through a delivery device or human tissue, and may be resorbed or otherwise removed after the intervertebral prosthetic disc 500 is in place. Also, the hydrophilic layers 640, 740 can be forced, or otherwise squeezed, from the bearing surfaces 606, 706 under the weight of the patient, after installation, to allow the keels 630, 632, 730, 732 and the teeth 634, 734 to engage the vertebra.

As shown in FIG. 7, the inferior hydrophilic layer 640 can have a thickness, or height, that is greater than a height of the inferior teeth 634, a height of the first inferior keel 630, and a height of the second inferior keel 632. As such, the inferior hydrophilic layer 640 can be hydrated in order to substantially prevent the inferior teeth 634 and the inferior keels 630, 632 from dragging along an interior surface of a delivery device. Further, the inferior hydrophilic layer 640 can prevent the inferior teeth 634 and the inferior keels 630, 632 from abrading human tissue during implantation of the intervertebral prosthetic disc 500.

Also, as depicted in FIG. 7, the superior hydrophilic layer 740 can have a thickness, or height, that is greater than a height of the superior teeth 734, a height of the first superior keel 730, and a height of the second superior keel 732. As such, the superior hydrophilic layer 740 can be hydrated in order to substantially prevent the superior teeth 734 and the superior keels 730, 732 from dragging along an interior surface of a delivery device. Further, the superior hydrophilic layer 740 can prevent the superior teeth 734 and the superior keels 730, 732 from abrading human tissue during implantation of the intervertebral prosthetic disc 500.

In a particular embodiment, the inferior hydrophilic layer 640, the superior hydrophilic layer 740, or a combination thereof, can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

Installation of a Spinal Implant Within an Intervertebral Space

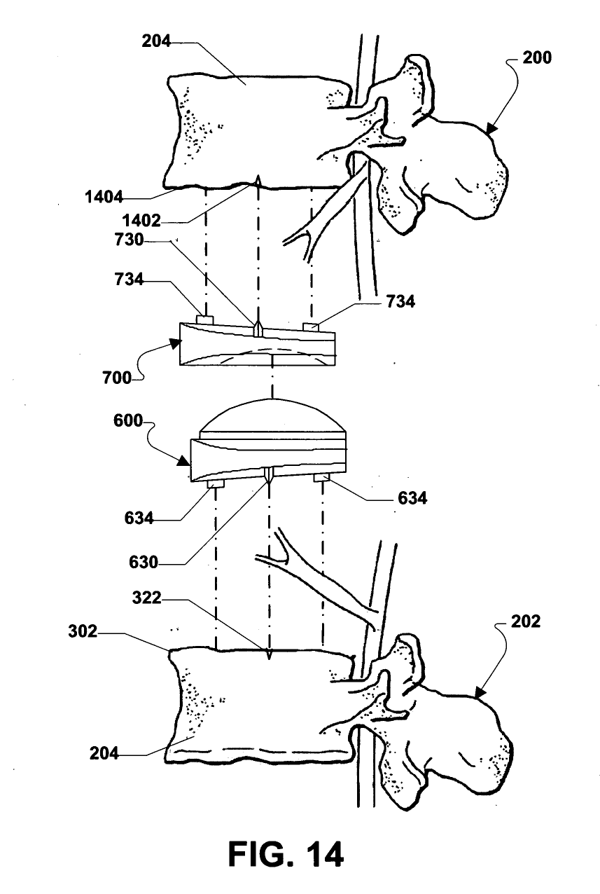

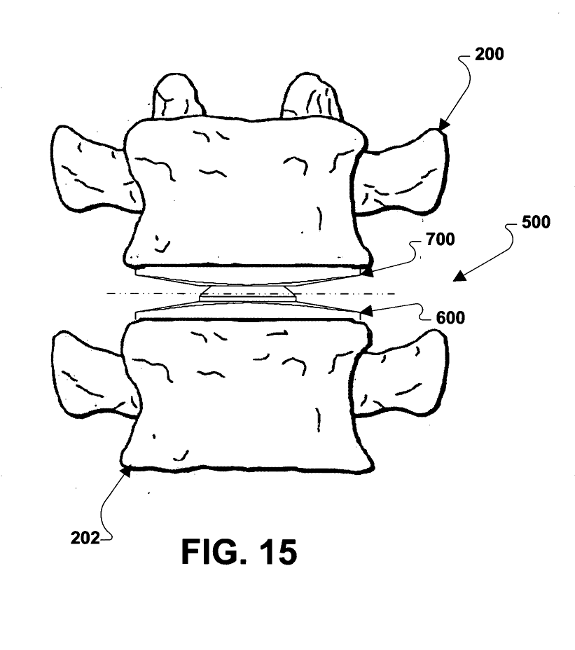

Referring to FIG. 14 and FIG. 15, an intervertebral prosthetic disc is shown between the superior vertebra 200 and the inferior vertebra 202, previously introduced and described in conjunction with FIG. 2 and FIG. 3. In a particular embodiment, the intervertebral prosthetic disc is the intervertebral prosthetic disc 500 described in conjunction with FIG. 5 through FIG. 13. Alternatively, the intervertebral prosthetic disc can be an intervertebral prosthetic disc according to any of the embodiments disclosed herein.

As shown in FIG. 14 and FIG. 15, the intervertebral prosthetic disc 500 is installed within the intervertebral space 214 that can be established between the superior vertebra 200 and the inferior vertebra 202 by removing vertebral disc material (not shown). FIG. 14 shows that the inferior teeth 634 of the inferior articular half 600 can engage the cancellous bone of the inferior vertebra 202. Further, the first inferior rib 630 of the inferior articular half 600 can engage a first slot 322 that can be established within the vertebral body 204 of the inferior vertebra 202. In particular, the first slot 322 can be established within the cortical rim 302 of the vertebral body 204 of the inferior vertebra 202. A second inferior rib (not shown in FIG. 14) of the inferior articular half 600 can engage a second slot (not shown in FIG. 14) that can be established within the vertebral body 204 of the inferior vertebra 202.

FIG. 14 also indicates that the superior teeth 734 of the superior articular half 700 can engage the cancellous bone of the superior vertebra 300. Moreover, the first superior rib 730 of the superior articular half 700 can engage a first slot 1402 that is established within the vertebral body 204 of the superior vertebra 200. In particular, the first slot 1402 can be established within the cortical rim 1404 of the vertebral body 204 of the superior vertebra 200. A second superior rib (not shown in FIG. 14) of the superior articular half 700 can engage a second slot (not shown in FIG. 14) that can be established within the vertebral body 204 of the superior vertebra 202.

As illustrated in FIG. 14 and FIG. 15, the projection 608 that extends from the inferior articular half 600 of the intervertebral prosthetic disc 500 can engage the depression 708 that is formed within the superior articular half 700 of the intervertebral prosthetic disc 500. It is to be appreciated that when the intervertebral prosthetic disc 500 is installed between the superior vertebra 200 and the inferior vertebra 202, the intervertebral prosthetic disc 500 allows relative motion between the superior vertebra 200 and the inferior vertebra 202. Specifically, the configuration of the inferior articular half 600 and the superior articular half 700 allows the inferior articular half 600 to rotate with respect to the superior articular half 700. As such, the superior vertebra 200 can rotate with respect to the inferior vertebra 202.

The intervertebral prosthetic disc 500 can be delivered to the intervertebral space 214 through a delivery device (not shown), such as an insertion tube, or the like, designed to deliver an intervertebral prosthetic disc to a point of use. The delivery device can be a closed tube having a cross-section that can be generally circular, generally rectangular, generally square, generally triangular, generally trapezoidal, generally rhombic, generally quadrilateral, any generally polygonal shape, or a combination thereof. Further, the delivery device can be an open channel having a cross-section that can be generally U-shaped, generally V-shaped, generally semi-circular, generally arcuate, generally box shaped, or a combination thereof.

Movement of the intervertebral prosthetic disc through the delivery device can be facilitated by a hydrophilic surface, which can be “activated” (i.e., made more lubricious) by exposing the same to a biocompatible fluid, such as a saline solution, natural or synthetic synovial fluid or the like. Additionally, the intervertebral prosthetic disc can be exposed to a patient's body fluid, e.g., fat, blood, or a combination thereof. Further, the intervertebral prosthetic disc can be exposed to any other synthetic or natural biocompatible fluid. Activation of the hydrophilic surface can occur before introducing the intervertebral prosthetic disc into the delivery device, such as by dipping in, spraying with or otherwise contacting the hydrophilic surface with an activating fluid. Alternatively or in addition, the hydrophilic surface can be activated after the intervertebral prosthetic disc is placed in the delivery device by introducing an activating fluid into the delivery device. Further, a hydrophilic surface on the intervertebral prosthetic disc can be activated proximate to the intervertebral space (e.g., by natural synovial fluid or the like) to aid insertion into the intervertebral space.

In a particular embodiment, the intervertebral prosthetic disc 500 can allow angular movement in any radial direction relative to the intervertebral prosthetic disc 500. Further, as depicted in FIG. 14 and 15, the inferior articular half 600 can be placed on the inferior vertebra 202 so that the center of rotation of the inferior articular half 600 is substantially aligned with the center of rotation of the inferior vertebra 202. Similarly, the superior articular half 700 can be placed relative to the superior vertebra 200 so that the center of rotation of the superior articular half 700 is substantially aligned with the center of rotation of the superior vertebra 200. Accordingly, when the vertebral disc, between the inferior vertebra 202 and the superior vertebra 200, is removed and replaced with the intervertebral prosthetic disc 500 the relative motion of the vertebrae 200, 202 provided by the vertebral disc is substantially replicated.

Description of a Nucleus Implant

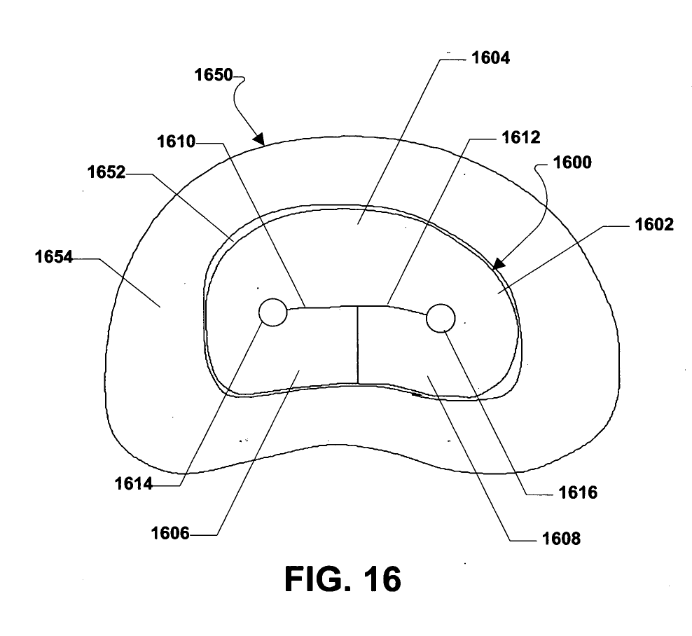

Referring to FIG. 16 through FIG. 19, an embodiment of a nucleus implant is shown and is designated 1600. As shown, the nucleus implant 1600 can include a load bearing elastic body 1602. The load bearing elastic body 1602 can include a central portion 1604. A first end 1606 and a second end 1608 can extend from the central portion 1604 of the load bearing elastic body 1602.

In a particular embodiment, the load bearing elastic body 1602 can be made from one or more biocompatible materials. For example, the biocompatible materials can be one or more polymer materials. The polymer materials can include polyurethane materials, polyolefin materials, polyaryletherketone materials, polyester materials, silicone materials, hydrogel materials, or a combination thereof. Further, the polyolefin materials can include polypropylene, polyethylene, halogenated polyolefin, flouropolyolefin, or a combination thereof. The polyaryletherketone (PAEK) materials can include polyetherketone (PEK), polyetheretherketone (PEEK), polyetherketoneketone (PEKK), polyetherketoneetherketoneketone (PEKEKK), or a combination thereof. The polyester materials include polylactide. The hydrogels can include polyacrylamide, poly-N-isopropylacrylamine, polyvinyl methylether, polyvinyl alcohol, polyethyl hydroxyethyl cellulose, poly(2-ethyl)oxazoline, polyethyleneoxide, polyethylglycol, polyethylene glycol, polyacrylic acid, polyacrylonitrile, polyvinylacrylate, polyvinylpyrrolidone, or a combination thereof.

Alternatively, the load bearing elastic body 1602 can be made from any other substantially elastic biocompatible materials.

As depicted in FIG. 16, the first end 1606 of the load bearing elastic body 1602 can establish a first fold 1610 with respect to the central portion 1604 of the load bearing elastic body 1602. Further, the second end 1608 of the load bearing elastic body 1602 can establish a second fold 1612 with respect to the central portion 1604 of the load bearing elastic body 1602. In a particular embodiment, the ends 1606, 1608 of the load bearing elastic body 1602 can be folded toward each other relative to the central portion 1604 of the load bearing elastic body 1602. Further, in a particular embodiment, the first fold 1610 can define a first aperture 1614 and the second fold 1612 can define a second aperture 1616. In a particular embodiment, the apertures 1614, 1616 are generally circular. However, the apertures 1614, 1616 can have any arcuate shape.

FIG. 16 indicates that the nucleus implant 1600 can be implanted within an intervertebral disc 1650 between a superior vertebra and an inferior vertebra. More specifically, the nucleus implant 1600 can be implanted within an intervertebral disc space 1652 established within the annulus fibrosus 1654 of the intervertebral disc 1650. The intervertebral disc space 1652 can be established by removing the nucleus pulposus (not shown) from within the annulus fibrosus 1654.

In a particular embodiment, the nucleus implant 1600 can provide shock-absorbing characteristics substantially similar to the shock absorbing characteristics provided by a natural nucleus pulposus. Additionally, in a particular embodiment, the nucleus implant 1600 can have a height that is sufficient to provide proper support and spacing between a superior vertebra and an inferior vertebra.

In a particular embodiment, the nucleus implant 1600 shown in FIG. 16 can have a shape memory and the nucleus implant 1600 can be configured to allow extensive short-term manual, or other, deformation without permanent deformation, cracks, tears, breakage or other damage, that may occur, for example, during placement of the implant into the intervertebral disc space 1652.

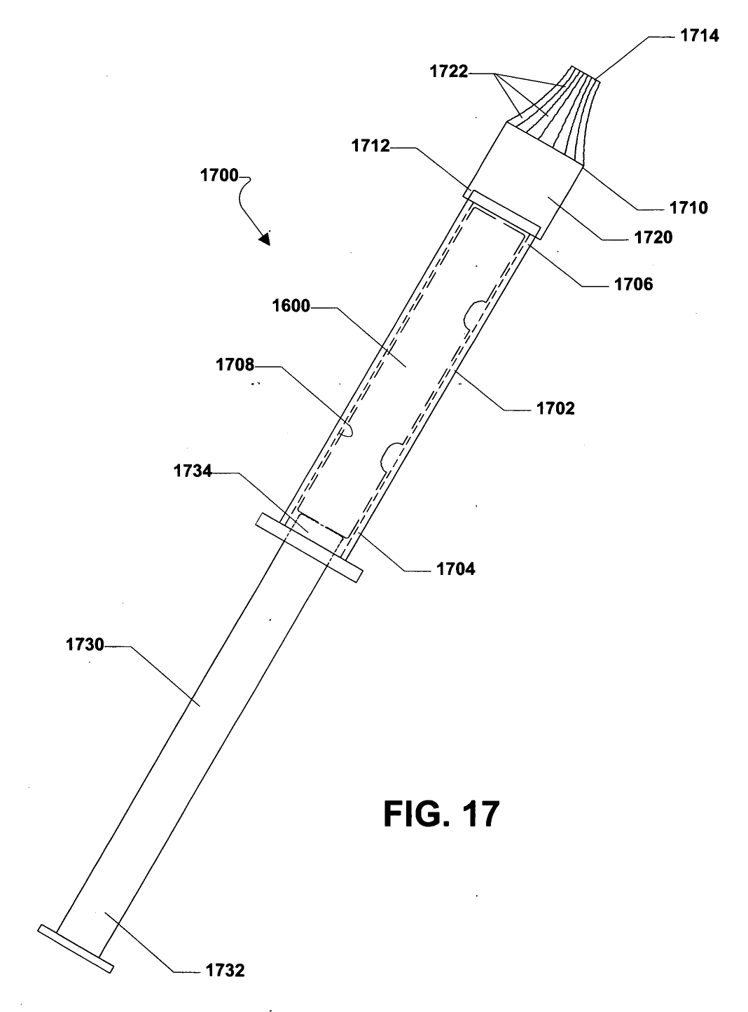

For example, the nucleus implant 1600 can be deformable, or otherwise configurable, e.g., manually, from a folded configuration, shown in FIG. 16, to a substantially straight configuration, shown in FIG. 17. In a particular embodiment, when the nucleus implant 1600 the folded configuration, shown in FIG. 16, can be considered a relaxed state for the nucleus implant 1600. Also, the nucleus implant 1600 can be placed in the straight configuration for placement, or delivery into an intervertebral disc space within an annulus fibrosis.

In a particular embodiment, the nucleus implant 1600 can include a shape memory, and as such, the nucleus implant 1600 can automatically return to the folded, or relaxed, configuration from the straight configuration after force is no longer exerted on the nucleus implant 1600. Accordingly, the nucleus implant 1600 can provide improved handling and manipulation characteristics since the nucleus implant 1600 can be deformed, configured, or otherwise handled, by an individual without resulting in any breakage or other damage to the nucleus implant 1600.

Although the nucleus implant 1600 can have a wide variety of shapes, the nucleus implant 1600 when in the folded, or relaxed, configuration can conform to the shape of a natural nucleus pulposus. As such, the nucleus implant 1600 can be substantially elliptical when in the folded, or relaxed, configuration. In one or more alternative embodiments, the nucleus implant 1600, when folded, can be generally annular-shaped or otherwise shaped as required to conform to the intervertebral disc space within the annulus fibrosis. Moreover, when the nucleus implant 1600 is in an unfolded, or non-relaxed, configuration, such as the substantially straightened configuration, the nucleus implant 1600 can have a wide variety of shapes. For example, the nucleus implant 1600, when straightened, can have a generally elongated shape. Further, the nucleus implant 1600 can have a cross section that is: generally elliptical, generally circular, generally rectangular, generally square, generally triangular, generally trapezoidal, generally rhombic, generally quadrilateral, any generally polygonal shape, or any combination thereof.

Referring to FIG. 17, an implant delivery device is shown and is generally designated 1700. As illustrated in FIG. 17, the implant delivery device 1700 can include an elongated housing 1702 that can include a proximal end 1704 and a distal end 1706. The elongated housing 1702 can be hollow and can form an internal cavity 1708. As depicted in FIG. 17, the implant delivery device 1700 can also include a tip 1710 having a proximal end 1712 and a distal end 1714. In a particular embodiment, the proximal end 1712 of the tip 1710 can be affixed, or otherwise attached, to the distal end 1706 of the housing 1702.

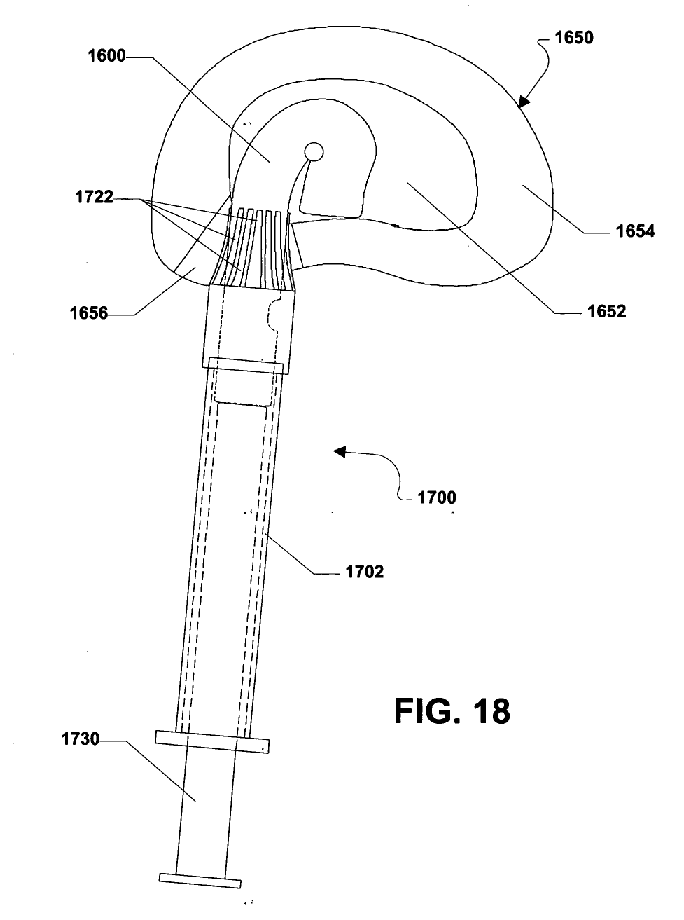

In a particular embodiment, the tip 1710 of the implant delivery device 1700 can include a generally hollow base 1720. Further, a plurality of movable members 1722 can be attached to the base 1720 of the tip 1710. The movable members 1722 are movable between a closed position, shown in FIG. 17, and an open position, shown in FIG. 18, as a nucleus implant is delivered using the implant delivery device 1700 as described below.

FIG. 17 further shows that the implant delivery device 1700 can include a generally elongated plunger 1730 that can include a proximal end 1732 and a distal end 1734. In a particular embodiment, the plunger 1730 can be sized and shaped to slidably fit within the housing 1702, e.g., within the cavity 1708 of the housing 1702.

As shown in FIG. 17 and FIG. 18, a nucleus implant, e.g., the nucleus implant 1600 shown in FIG. 16, can be disposed within the housing 1702, e.g., within the cavity 1708 of the housing 1702. Further, the plunger 1730 can slide within the cavity 1708, relative to the housing 1702, in order to force the nucleus implant 1600 from within the housing 1702 and into the intervertebral disc space 1652. As shown in FIG. 18, as the nucleus implant 1600 exits the implant delivery device 1700, the nucleus implant 1600 can move from the non-relaxed, straight configuration to the relaxed, folded configuration within the annulus fibrosis. Further, as the nucleus implant 1600 exits the implant delivery device 1700, the nucleus implant 1600 can cause the movable members 1722 to move to the open position, as shown in FIG. 18.

In a particular embodiment, the nucleus implant 1600 can be installed using a posterior surgical approach, as shown. Further, the nucleus implant 1600 can be installed through a posterior incision 1656 made within the annulus fibrosus 1654 of the intervertebral disc 1650. Alternatively, the nucleus implant 1600 can be installed using an anterior surgical approach, a lateral surgical approach, or any other surgical approach well known in the art.

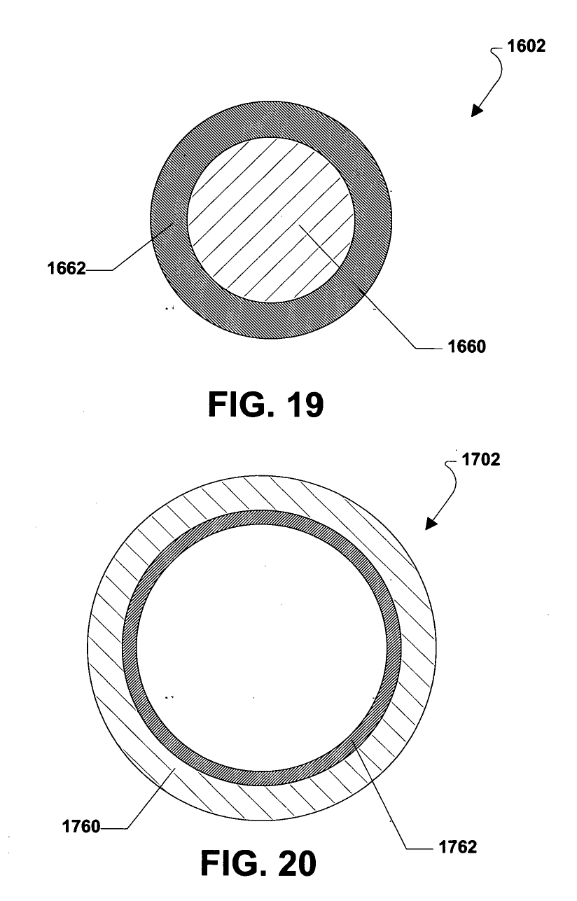

Referring to FIG. 19, the load bearing elastic body 1602 is illustrated in cross-section. As shown, the load bearing elastic body 1602 can include a core 1660 and an outer hydrophilic layer 1662 that can surround the core 1660. In a particular embodiment, the core 1660 of the load bearing elastic body can be made from one or more biocompatible materials. For example, the biocompatible materials can be one or more polymer materials, described herein.

In a particular embodiment, the load bearing elastic body 1602 can be treated to increase the hydrophilicity of the load bearing elastic body 1602. Specifically, the external surfaces of the load bearing elastic body 1602 can be treated to establish the outer hydrophilic layer 1662 that is more hydrophilic than the underlying material that is used to make the load bearing elastic body 1602.

For example, the outer hydrophilic layer 1662 of the load bearing elastic body 1602 can be formed by oxidizing the outer surfaces of the load bearing elastic body 1602. Additionally, the outer hydrophilic layer 1662 of the load bearing elastic body 1602 can be formed using a chemical technique or an electrochemical technique.

In a particular embodiment, the chemical technique or the electrochemical technique can include a gas plasma technique. In other words, the elastic body 1602 can be exposed to a gas plasma in order to modify the hydrophilicity or wettability of the surface of the elastic body 1602. For example, the surface of the elastic body 1602 can be modified using a cold gas plasma process. The cold gas plasma process can include placing the nucleus implant 1600 in a vacuum and pumping in one or more process fluids. Radio-frequency energy can be supplied to one or more electrodes within the chamber in order to excite the process fluid into plasma.

The process fluid can include one or more gases, one or more liquids, or a combination thereof. Further, the one or more gases can include oxygen, argon, helium, nitrogen, ammonia, hydrogen, nitrous oxide, carbon dioxide, air, methane, ethane, ethylene, acetylene, tetrafluoromethane, hexafluoroethane, hexafluoropropylene, or combination thereof. Moreover, the one or more liquids can include methanol, water, allyl amine, ethylenediamine, acrylic acide, acetone, hydroxyethylmethacrylate, ethanol, toluene, diaminopropane, butylamine, gluteraldehyde, hexamethyldisiloxane, tetramethylsilane, polyethylene glycol, diglyme, silane, or a combination thereof.

In a particular embodiment, the outer hydrophilic layer 1662 can be one or more hydrophilic polymers that can be surface grafted on the core 1660 of the load bearing elastic body 1602.

The hydrophilic polymers can include polyalkylene glycol, polymethacrylates, maleic anhydride/vinyl ether copolymer, starch, starch derivatives, gelatin, alginate, hydroxyethyl methacrylate, carrageenan, polyurethane, agar, carboxyvinyl copolymer, polyethylene oxide, polyhydroxyethyl methacrylate, polydioxolane, polyacryl acetate, polyvinyl chloride, or a combination thereof.

Further, the hydrophilic polymers can include one or more cellulose derivative, such as hydroxypropylmethyl cellulose, hydroxypropyl cellulose, carboxymethyl cellulose, methyl cellulose, ethyl cellulose, hydroxyethyl cellulose, hydroxymethyl cellulose, carboxyethylcellulose, carboxy-methyl-hydroxy-ethyl cellulose, or a combination thereof.

In another particular embodiment, the outer hydrophilic layer 1662 can be one or more hydrogels. The hydrogels can include polyacrylamide, poly-N-isopropylacrylamine, polyvinyl methylether, polyvinyl alcohol, polyethyl hydroxyethyl cellulose, poly(2-ethyl)oxazoline, polyethyleneoxide, polyethylglycol, polyethylene glycol, polyacrylic acid, polyacrylonitrile, polyvinylacrylate, polyvinylpyrrolidone, or a combination thereof.

In a particular embodiment, the outer hydrophilic layer 1660 can be resorbable, non-resorbable, temporary, permanent, semi-permanent, detachable, removable, or a combination thereof. For example, the outer hydrophilic layer 1660 may provide lubrication during installation and may be resorbed or otherwise removed after the nucleus implant 1600 is installed.

In a particular embodiment, the outer hydrophilic layer 1660 can be coated with, impregnated with, or otherwise include, a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

Referring now to FIG. 20, the housing 1702 is illustrated in cross-section. As shown, the housing 1702 can include an outer structure 1760 and an inner hydrophilic layer 1762 that can surround the outer structure 1760. In a particular embodiment, the outer structure 1760 of the load bearing elastic body can be made from one or more biocompatible materials. For example, the biocompatible materials can be one or more polymer materials, described herein.

In a particular embodiment, the housing 1702 can be treated to increase the hydrophilicity of the housing 1702. Specifically, the internal surface of the housing 1702 can be treated to establish the inner hydrophilic layer 1762 that is more hydrophilic than the underlying material that is used to make the housing 1702.

For example, the inner hydrophilic layer 1762 of the housing 1702 can be formed by oxidizing the inner surfaces of the housing 1702. Additionally, the inner hydrophilic layer 1762 of the housing 1702 can be formed using a chemical technique or an electrochemical technique.

In a particular embodiment, the chemical technique or the electrochemical technique can include a gas plasma technique. In other words, the inner surface of the housing 1702 can be exposed to a gas plasma in order to modify the hydrophilicity or wettability of the inner surface of the housing 1702. For example, the inner surface of the housing 1702 can be modified using a cold gas plasma process. The cold gas plasma process can include placing the implant delivery device 1700 in a vacuum and pumping in one or more process fluids. Radio-frequency energy can be supplied to one or more electrodes within the chamber in order to excite the process fluid into plasma.

The process fluid can include one or more gases, one or more liquids, or a combination thereof. Further, the one or more gases can include oxygen, argon, helium, nitrogen, ammonia, hydrogen, nitrous oxide, carbon dioxide, air, methane, ethane, ethylene, acetylene, tetrafluoromethane, hexafluoroethane, hexafluoropropylene, or combination thereof. Moreover, the one or more liquids can include methanol, water, allyl amine, ethylenediamine, acrylic acide, acetone, hydroxyethylmethacrylate, ethanol, toluene, diaminopropane, butylamine, gluteraldehyde, hexamethyldisiloxane, tetramethylsilane, polyethylene glycol, diglyme, silane, or a combination thereof.

In a particular embodiment, the inner hydrophilic layer 1762 can be one or more hydrophilic polymers that can be surface grafted on the outer-structure 1760 of the housing 1702.

The hydrophilic polymers can include polyalkylene glycol, polymethacrylates, maleic anhydride/vinyl ether copolymer, starch, starch derivatives, gelatin, alginate, hydroxyethyl methacrylate, carrageenan, polyurethane, agar, carboxyvinyl copolymer, polyethylene oxide, polyhydroxyethyl methacrylate, polydioxolane, polyacryl acetate, polyvinyl chloride, or a combination thereof.

Further, the hydrophilic polymers can include one or more cellulose derivative, such as hydroxypropylmethyl cellulose, hydroxypropyl cellulose, carboxymethyl cellulose, methyl cellulose, ethyl cellulose, hydroxyethyl cellulose, hydroxymethyl cellulose, carboxyethylcellulose, carboxy-methyl-hydroxy-ethyl cellulose, or a combination thereof.

In another particular embodiment, the inner hydrophilic layer 1762 can be one or more hydrogels. The hydrogels can include polyacrylamide, poly-N-isopropylacrylamine, polyvinyl methylether, polyvinyl alcohol, polyethyl hydroxyethyl cellulose, poly(2-ethyl)oxazoline, polyethyleneoxide, polyethylglycol, polyethylene glycol, polyacrylic acid, polyacrylonitrile, polyvinylacrylate, polyvinylpyrrolidone, or a combination thereof.

In a particular embodiment, the inner hydrophilic layer 1760 can be resorbable, non-resorbable, temporary, permanent, semi-permanent, detachable, removable, or a combination thereof. For example, the inner hydrophilic layer 1760 may provide lubrication during installation and may be removed after the nucleus implant 1600 is installed.

Description of a Method of Installing a Spinal Implant

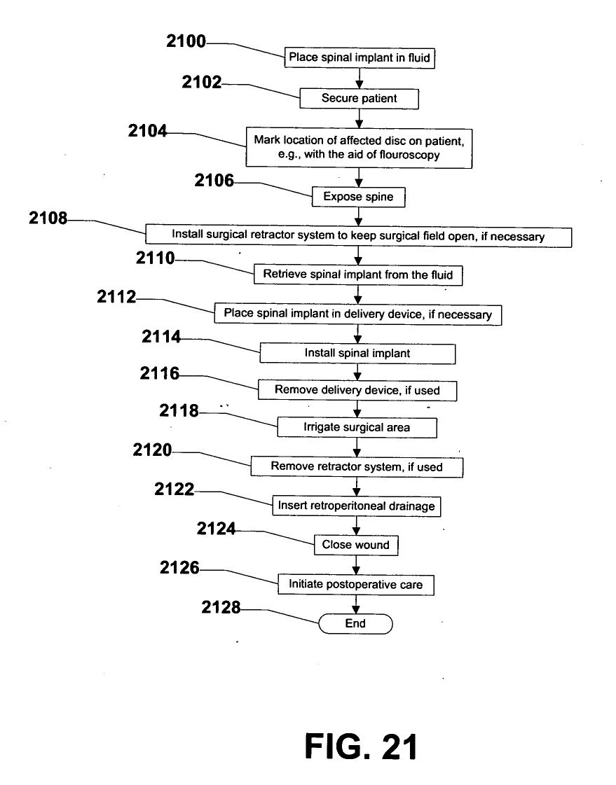

Referring to FIG. 21, an exemplary, non-limiting embodiment of a method of installing a nucleus implant is shown and commences at block 2100. At block 2100, a spinal implant is placed in a fluid. In a particular embodiment, the spinal implant is soaked in the fluid for a predetermined time. Alternatively, a hydrophilic surface or a hydrophilic layer of the spinal implant is exposed to the fluid. The fluid can be water, saline, blood, body fat, or a combination thereof. Moving to block 2102, a patient is secured on an operating table: For example, the patient can be secured in a supine position to allow an anterior approach to be used to access the patient's spinal column. Further, the patient may be placed in a “French” position in which the patient's legs are spread apart. The “French” position can allow the surgeon to stand between the patient's legs. Further, the “French” position can facilitate proper alignment of the surgical instruments with the patient's spine. In another particular embodiment, the patient can be secured in the supine position on an adjustable surgical table.

In one or more alternative embodiments, a surgeon can use a posterior approach or a lateral approach to implant an intervertebral prosthetic device. As such, the patient may be secured in a different position, e.g., in a prone position for a posterior approach or in a lateral decubitus position for a lateral approach.

Moving to block 2104, the location of the affected disc is marked on the patient, e.g., with the aid of fluoroscopy. At block 2106, the surgical area along spinal column is exposed. Further, at block 2108, a surgical retractor system can be installed to keep the surgical field open, if necessary. For example, the surgical retractor system can be a Medtronic Sofamor Danek Endoring™ Surgical Retractor System. In an alternative embodiment, the surgical technique used to access the spinal column may be a “keyhole” technique and a retractor system may not be necessary.

Continuing to block 2110, the spinal implant can be retrieved from the fluid. At block 2112, the spinal implant can be placed in a delivery device, if a delivery device is being used. In a particular embodiment, the spinal implant can be placed in the delivery device so that a hydrophilic surface, or a hydrophilic layer, at least partially contacts an interior surface of the delivery device. Thereafter, at block 2114, the spinal implant can be installed. At block 2116, the delivery device can be removed—if used.

Proceeding to block 2118, the surgical area can be irrigated. Further, at block 2120, the retractor system can be removed—if used. At block 2122, a drainage, e.g., a retroperitoneal drainage, can be inserted into the wound. Additionally, at block 2124, the surgical wound can be closed. The surgical wound can be closed using sutures, surgical staples, or any other surgical technique well known in the art. Moving to block 2126, postoperative care can be initiated. The method ends at state 2128.

Description of Another Method of Installing a Spinal Implant

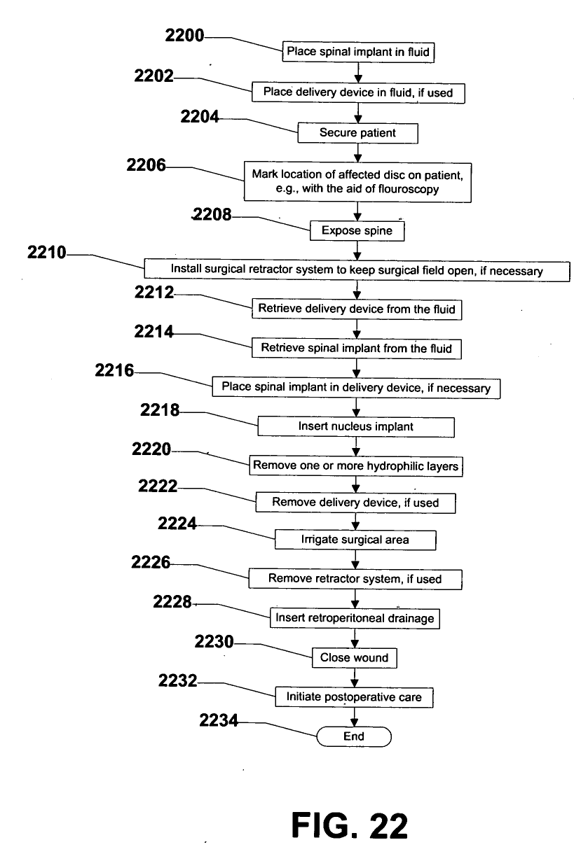

Referring to FIG. 22, an exemplary, non-limiting embodiment of a method of installing a nucleus implant is shown and commences at block 2200. At block 2200, a spinal implant can be placed in a fluid. At block 2202, a delivery device, if used, can also be placed in a fluid. The fluid can be water, saline, blood, body fat, or a combination thereof. Moving to block 2204, a patient is secured on an operating table. For example, the patient can be secured in a supine position to allow an anterior approach to be used to access the patient's spinal column. Further, the patient may be placed in a “French” position in which the patient's legs are spread apart. The “French” position can allow the surgeon to stand between the patient's legs. Further, the “French” position can facilitate proper alignment of the surgical instruments with the patient's spine. In another particular embodiment, the patient can be secured in the supine position on an adjustable surgical table.

In one or more alternative embodiments, a surgeon can use a posterior approach or a lateral approach to implant an intervertebral prosthetic device. As such, the patient may be secured in a different position, e.g., in a prone position for a posterior approach or in a lateral decubitus position for a lateral approach.

Moving to block 2206, the location of the affected disc is marked on the patient, e.g., with the aid of fluoroscopy. At block 2208, the surgical area along spinal column is exposed. Further, at block 2210, a surgical retractor system can be installed to keep the surgical field open, if necessary. For example, the surgical retractor system can be a Medtronic Sofamor Danek Endoring™ Surgical Retractor System. In an alternative embodiment, the surgical technique used to access the spinal column may be a “keyhole” technique and a retractor system may not be necessary.

Continuing to block 2212, the delivery device can be retrieved from the fluid. At block 2214, the spinal implant can be retrieved from the fluid. At block 2216, the spinal implant can be placed in a delivery device, if a delivery device is being used. Thereafter, at block 2218, the spinal implant can be installed. At block 2220, one or more hydrophilic layers can be removed from the spinal implant. Further, at block 2222, the delivery device can be removed—if used.

Proceeding to block 2224, the surgical area can be irrigated. Further, at block 2226, the retractor system can be removed—if used. At block 2228, a drainage, e.g., a retroperitoneal drainage, can be inserted into the wound. Additionally, at block 2230, the surgical wound can be closed. The surgical wound can be closed using sutures, surgical staples, or any other surgical technique well known in the art. Moving to block 2232, postoperative care can be initiated. The method ends at state 2234.

Conclusion

With the configuration of structure described above, the spinal implant according to one or more of the embodiments provides a device that may be implanted to replace a natural intervertebral disc that is diseased, degenerated, or otherwise damaged. The spinal implant can be disposed within an intervertebral disc space that can be established between an inferior vertebra and a superior vertebra. Alternatively, the spinal implant can be disposed within an intervertebral disc space that can be established within an intervertebral disc by removing the nucleus pulposus.

Further, after a patient fully recovers from a surgery to implant the spinal implant, the spinal implant can provide relative motion between the inferior vertebra and the superior vertebra that closely replicates the motion provided by a natural intervertebral disc. Accordingly, the spinal implant provides an alternative to a fusion device that can be implanted within the intervertebral space between the inferior vertebra and the superior vertebra to fuse the inferior vertebra and the superior vertebra and prevent relative motion there between.

In a particular embodiment, the spinal implant can be treated, as described herein, to increase the hydrophilicity of the spinal implant. Accordingly, when the spinal implant comes in contact with a fluid, e.g., saline, body fluid, another fluid, or a combination thereof, the spinal implant can retain the fluid and the spinal implant can become lubricated. Such lubrication can ease implant delivery, reduce tissue trauma during insertion, increase implant biocompatibility, and improve in vivo implant performance.

When lubricated, a coefficient of friction between the surface of the spinal implant and the interior surface of a delivery device can be substantially less than a coefficient of friction between an unlubricated surface and the interior surface of the delivery device. Further, a coefficient of friction between the surface of the spinal implant and human tissue can be substantially less than a coefficient of friction between the unlubricated surface and the human tissue.

Further, other spinal implants, not illustrated and described in detail herein, can be treated as described herein to increase the hydrophilicity of those implants. Such spinal implants can include nucleus replacement implants, annulus repairing devices, total disc prostheses, interspinous process spacers, facet replacement implants, interbody fusion cages, bone screws, spinal plates, spinal rods, spinal tethers, etc. Further, such implants can include implants of varying shapes and can include a sphere, a hemisphere, a solid ellipse, a cube, a cylinder, a pyramid, a prism, a rectangular solid shape, a cone, a frustum, or a combination thereof.

Also, these implants can be delivered through a delivery device having various shapes. The delivery device can be a closed tube having a cross-section that can be generally circular, generally rectangular, generally square, generally triangular, generally trapezoidal, generally rhombic, generally quadrilateral, any generally polygonal shape, or a combination thereof. Further, the delivery device can be an open channel having a cross-section that can be generally U-shaped, generally V-shaped, generally semi-circular, generally arcuate, generally box shaped, or a combination thereof. The delivery device can also be treated as described herein to increase the hydrophilicity of the delivery device. For example, the delivery device can include an interior surface that can be treated as described herein to increase the hydrophilicity of the interior surface of the delivery device. As such, the delivery device can be exposed to a fluid in order to increase the lubrication of the interior surface of the delivery device in order to ease passage of an implant through the delivery device.

The above-disclosed subject matter is to be considered illustrative, and not restrictive, and the appended claims are intended to cover all such modifications, enhancements, and other embodiments that fall within the true spirit and scope of the present invention. For example, it is noted that the components in the exemplary embodiments described herein are referred to as “superior” and “inferior” for illustrative purposes only and that one or more of the features described as part of or attached to a respective half may be provided as part of or attached to the other half in addition or in the alternative. Thus, to the maximum extent allowed by law, the scope of the present invention is to be determined by the broadest permissible interpretation of the following claims and their equivalents, and shall not be restricted or limited by the foregoing detailed description.

1. An intervertebral prosthetic disc configured to be installed within an intervertebral space between a superior vertebra and an inferior vertebra, the intervertebral prosthetic disc comprising:
a superior component; and
an inferior component having an inferior bearing surface, wherein a hydrophilicity of the inferior bearing suite is greater than an average hydrophilicity of the inferior component.

2. The intervertebral prosthetic disc of claim 1, wherein the inferior bearing surface is configured to decrease a coefficient of friction of the inferior bearing surface when exposed to a fluid.

3. The intervertebral prosthetic disc of claim 1, wherein the inferior component further comprises an inferior hydrophilic layer thereon.

4. The intervertebral prosthetic disc of claim 3, wherein the inferior hydrophilic layer is substantially resorbable.

5. The intervertebral prosthetic disc of claim 3, wherein the inferior hydrophilic layer is substantially non-resorbable.

6. The intervertebral prosthetic disc of claim 3, wherein the inferior hydrophilic layer is temporary.

7. The intervertebral prosthetic disc of claim 3, wherein the inferior hydrophilic layer is substantially permanent.

8. The intervertebral prosthetic disc of claim 3, wherein the inferior hydrophilic layer is semi-permanent.

9. The intervertebral prosthetic disc of claim 3, wherein the inferior hydrophilic layer is detachable.

10. The intervertebral prosthetic disc of claim 3, wherein the inferior hydrophilic layer comprises a hydrophilic polymer.

11. The intervertebral prosthetic disc of claim 10, wherein the hydrophilic polymer comprises polyalkylene glycol, polymethacrylates, maleic anhydride/vinyl ether copolymer, starch, starch derivatives, gelatin, alginate, hydroxyethyl methacrylate, carrageenan, polyurethane, agar, carboxyvinyl copolymer, polyethylene oxide, polyhydroxyethyl methacrylate, polydioxolane, polyacryl acetate, polyvinyl chloride, hydroxypropylmethyl cellulose, hydroxypropyl cellulose, carboxymethyl cellulose, methyl cellulose, ethyl cellulose, hydroxyethyl cellulose, hydroxymethyl cellulose, carboxyethylcellulose, carboxy-methyl-hydroxyethyl cellulose, or a combination thereof

12. The intervertebral prosthetic disc of claim 3, wherein the inferior hydrophilic layer comprises a hydrogel.

13. The intervertebral prosthetic disc of claim 12, wherein the hydrogel comprises polyacrylamide, poly-N-isopropylacrylamine, polyvinyl methylether, polyvinyl alcohol, polyethyl hydroxyethyl cellulose, poly(2-ethyl)oxazoline, polyethyleneoxide, polyethylglycol, polyethylene glycol, polyacrylic acid, polyacrylonitrile, polyvinylacrylate, polyvinylpyrrolidone, or a combination thereof

14. The intervertebral prosthetic disc of claim 1, wherein the superior component includes a superior bearing surface having a hydrophilicity greater than an average hydrophilicity of the superior component.

15. The intervertebral prosthetic disc of claim 14, wherein the superior bearing surface is configured to decrease a coefficient of friction of the superior bearing surface when exposed to a fluid.

16. The intervertebral prosthetic disc of claim 14, wherein the superior component further comprises an superior hydrophilic layer thereon.

17. The intervertebral prosthetic disc of claim 16, wherein the superior hydrophilic layer is substantially resorbable.

18. The intervertebral prosthetic disc of claim 16, wherein the superior hydrophilic layer is substantially non-resorbable.

19. The intervertebral prosthetic disc of claim 16, wherein the superior hydrophilic layer is temporary.

20. The intervertebral prosthetic disc of claim 16, wherein the superior hydrophilic layer is substantially permanent.

21. The intervertebral prosthetic disc of claim 16, wherein the superior hydrophilic layer is semi-permanent.

22. The intervertebral prosthetic disc of claim 16, wherein the superior hydrophilic layer is detachable.

23. The intervertebral prosthetic disc of claim 16, wherein the superior hydrophilic layer comprises a hydrophilic polymer.

24. The intervertebral prosthetic disc of claim 16, wherein the hydrophilic polymer comprises polyalkylene glycol, polymethacrylates, maleic anhydride/vinyl ether copolymer, starch, starch derivatives, gelatin, alginate, hydroxyethyl methacrylate, carrageenan, polyurethane, agar, carboxyvinyl copolymer, polyethylene oxide, polyhydroxyethyl methacrylate, polydioxolane, polyacryl acetate, polyvinyl chloride, hydroxypropylmethyl cellulose, hydroxypropyl cellulose, carboxymethyl cellulose, methyl cellulose, ethyl cellulose, hydroxyethyl cellulose, hydroxymethyl cellulose, carboxyethylcellulose, carboxy-methyl-hydroxy-ethyl cellulose, or a combination thereof.

25. The intervertebral prosthetic disc of claim 16, wherein the superior hydrophilic layer comprises a hydrogel.

26. The intervertebral prosthetic disc of claim 25, wherein the hydrogel comprises polyacrylamide, poly-N-isopropylacrylamine, polyvinyl methylether, polyvinyl alcohol, polyethyl hydroxyethyl cellulose, poly(2-ethyl)oxazoline, polyethyleneoxide, polyethylglycol, polyethylene glycol, polyacrylic acid, polyacrylonitrile, polyvinylacrylate, polyvinylpyrrolidone, or a combination thereof.

27. A nucleus implant configured to be installed within an intervertebral space within an intervertebral disc comprising:
a load bearing elastic body movable between a folded configuration and a substantially straight configuration, wherein the load bearing elastic body has a core and an outer hydrophilic layer around the core.

28-39. (canceled)

40. A method of installing a spinal implant having a hydrophilic surface, the method comprising,
exposing the hydrophilic surface to a fluid; and
installing the spinal implant.

41-56. (canceled)

57. An implant delivery device, comprising:
a housing having an outer structure and an inner hydrophilic layer thereon.

58-63. (canceled)

64. A method of installing a spinal implant, the method comprising the steps of.
decreasing a coefficient of friction of the spinal implant; and
installing the implant.

65-76. (canceled)
